(19) United States
(12) Patent Application Publication
(10) Pub. No.: US 2004/0044346 A1
(43) Pub. Date: Mar. 4, 2004

(54) Surgical tool with disposable/removable cutting tip

(76) Inventor: Harb N. Boury, Glen Ellyn, IL (US)

Correspondence Address: Thomas G. Scavone, Niro, Scavone, Haller & Niro, 181 West Madison Street Suite 4600, Chicago, IL, 60602, US

(21) Appl. No.: 10/233,923

(22) Filed: Sep. 3, 2002

(51) Int. Cl. A61B 17/16

(52) U.S. Cl. 606/83

(57) ABSTRACT

The present invention concerns a rongeur which includes a moveable arm and a stationary arm linked in a sliding relationship by a pair of spaced apart guides located on one of the arms and a pair of spaced apart passageways located on the other of the arms. A stationary handle is connected to the stationary arm and a moveable handle is coupled to the moveable arm to permit the moveable arm to operate between open and closed positions. Also provided are at least two removable cutting tips which are releasably secured to the arms. The tips may be removed for sharpening and replaced by a replacement tip.

### BACKGROUND OF THE INVENTION

The present invention relates generally to a medical device having one or more replaceable cutting tips. More specifically, the present invention relates to a rongeur that has at least one replaceable cutting tip. In another embodiment, the tip or tips may be disposable.

### SUMMARY OF THE INVENTION

The rongeur is a surgical tool commonly used in performing spinal surgery. A key to the safe and efficient use of a rongeur is the sharpness of the opposing biting or cutting ends. Because they are used to remove bone, cartilage and other fibrous tissue, the cutting surfaces may dull prematurely, diminishing the utility of the device, and increasing the surgical risks.

To address this problem it is necessary periodically, actually quite frequently, to sharpen the cutting surfaces of the rongeur. The sharpening is typically performed by an outside service which requires that the entire device be delivered to this outside service and then returned. This procedure is not only costly, it may also require a few days to even weeks before the resharpened rongeur is returned. Moreover, the resharpened tool may not be as sharp as a new rongeur which may result in further delay in its availability. As a consequence, the neuro-surgical team at a particular medical facility or hospital may require several sets of these relatively expensive tools. Indeed, repeated sharpening may result in slightly displaced or non-symmetrically cutting surfaces so that approximation and/or alignment of the cutting surfaces is adversely affected. Ultimately then, the entire rongeur is rendered unuseable.

The present invention overcomes the drawbacks mentioned above by providing a rongeur that has one or more replaceable cutting surfaces. To facilitate the removal, as well as to securely retain the tips, the tips may be attached through the use of threaded fasteners as well as other interlocking mechanisms. In this way, the turnaround time required for a rongeur with dulled cutting surfaces can be reduced dramatically, while maintaining excellent sharpness of the tool's cutting surfaces at all times. In addition, the number of sets of rongeurs required at a given facility may be reduced.

### DESCRIPTION OF THE DRAWINGS

These and other features, objects and advantages of the present invention will become apparent from the following description and drawings wherein like reference numerals represent like elements in several views, and in which:

FIG. 1 shows a standard prior art rongeur without replaceable tips.

FIG. 2 shows a partial exploded view of the rongeur shown in FIG. 1.

FIG. 3 shows a partial exploded view of one embodiment of the present invention.

FIG. 4 shows a partial exploded view illustrating how cutting tips may be replaced.

FIG. 5 is a top view of one embodiment the present invention.

FIG. 6 is a cross-sectional view taken along line 6-6 in FIG. 5.

FIG. 7 is a top view of another embodiment of the present invention.

FIG. 8 is a cross-sectional view of the embodiment shown in FIG. 7 taken along line 8-8.

FIG. 9 is a partial perspective view of an alternate embodiment of the present invention.

FIG. 10 is a partial perspective view of yet another embodiment of the present invention.

FIG. 11 is a partial perspective view of still another embodiment of the present invention.

FIG. 12 is a partial, exploded view of another embodiment of the present invention.

FIG. 13 is a top view of the embodiment shown in FIG. 12.

FIG. 14 is a partial perspective view of the embodiment shown in FIG. 12.

FIG. 15 is a partial, exploded view of another embodiment of the present invention.

FIG. 16 is a cross-sectional view of the embodiment shown in FIG. 15 taken along line 16-16.

FIG. 17 illustrates how the sections of the embodiment shown in FIG. 15 inter-connect.

FIG. 18 illustrates another embodiment of the present invention.

FIG. 19 is a sectional view of the embodiment shown in FIG. 18 taken a long line 19-19.

FIG. 20 is a sectional view of the embodiment shown in FIG. 18 taken along line 20-20.

### DESCRIPTION OF THE PREFERRED EMBODIMENTS

Set forth below is a description of what are currently believed to be the preferred embodiments or best examples of the invention claimed. Future and present alternatives and modifications to the preferred embodiments are contemplated. Any alternates or modifications in which insubstantial changes in function, in purpose, in structure or in result are intended to be covered by the claims of this patent.

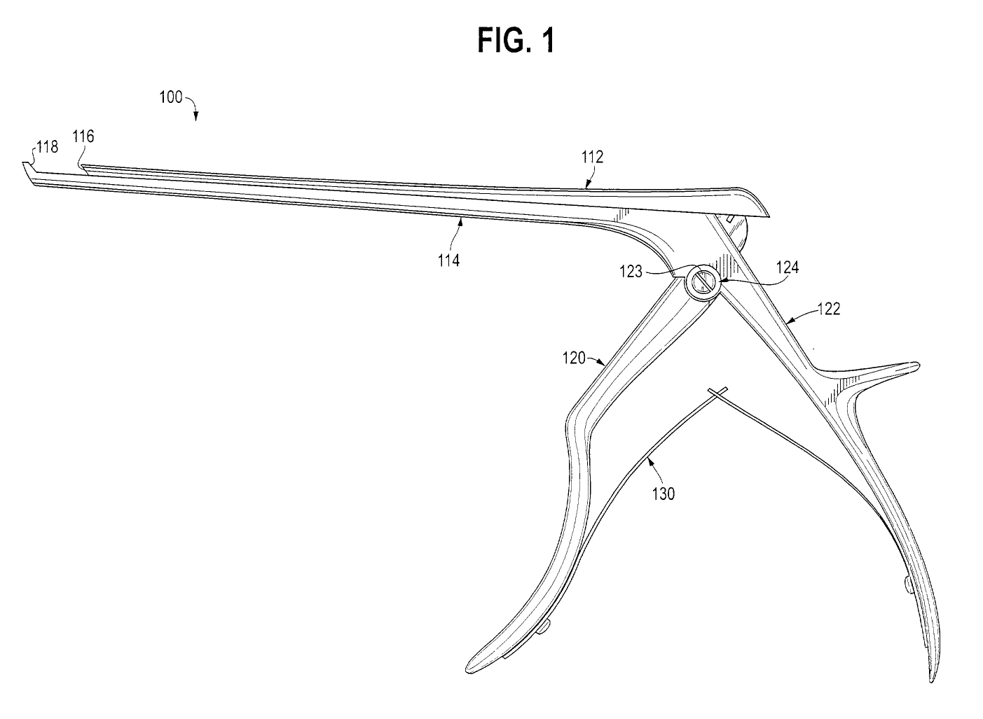

FIG. 1 illustrates a standard prior art rongeur 100 that does not include replaceable or removable tips. The device includes an elongated upper cutting arm 112 that moves between an open and closed position. A second, stationary arm 114 is also provided. At the distal ends of the arms are sharpened sections 116 and 118 that are cupped or concave in construction. Sections 116 and 118 provide the ability to cut bone, cartilage or other fibrous tissue.

A moveable handlebar 120 is attached to stationary handlebar 122 at pivot point 124 by a fastener 123. Handlebar 120 is coupled to arm 112 so as to urge the arm into a closed position to effectuate cutting. A spring 130 urges arm 112 into an open position by biasing handlebar 120 away from handlebar 122.

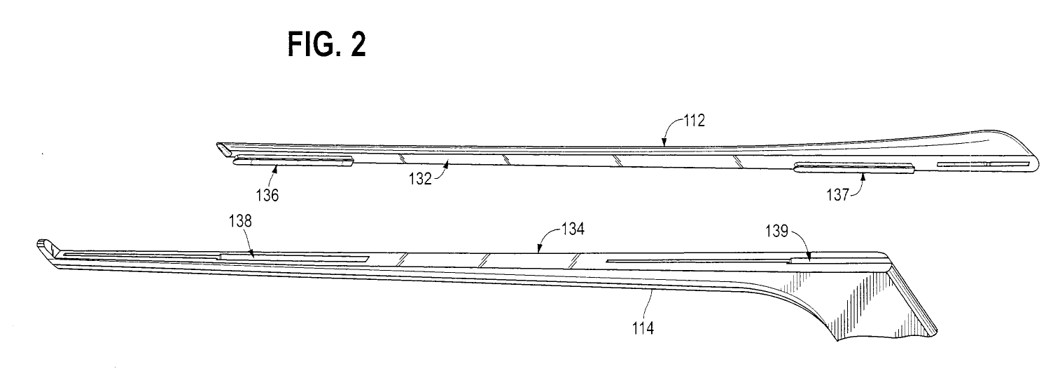

As shown in FIG. 2, arm 112 further includes a flat section 132 that engages or abuts flat section 134 of arm 114 to facilitate smooth operation of arm 112 with respect to arm 114. To link the arms in a sliding relationship, keys or guides 136 and 137 are configured to seat within keyways or passageways 138 and 139. Of course, the guides may be located on arm 114 and the passageways on arm 112.

To service or sharpen the cutting sections, the entire device is disassembled by the removal of screw 123 which allows the device to be broken down into its component parts. Of course, prior to operation the entire device needs to be re-assembled as well.

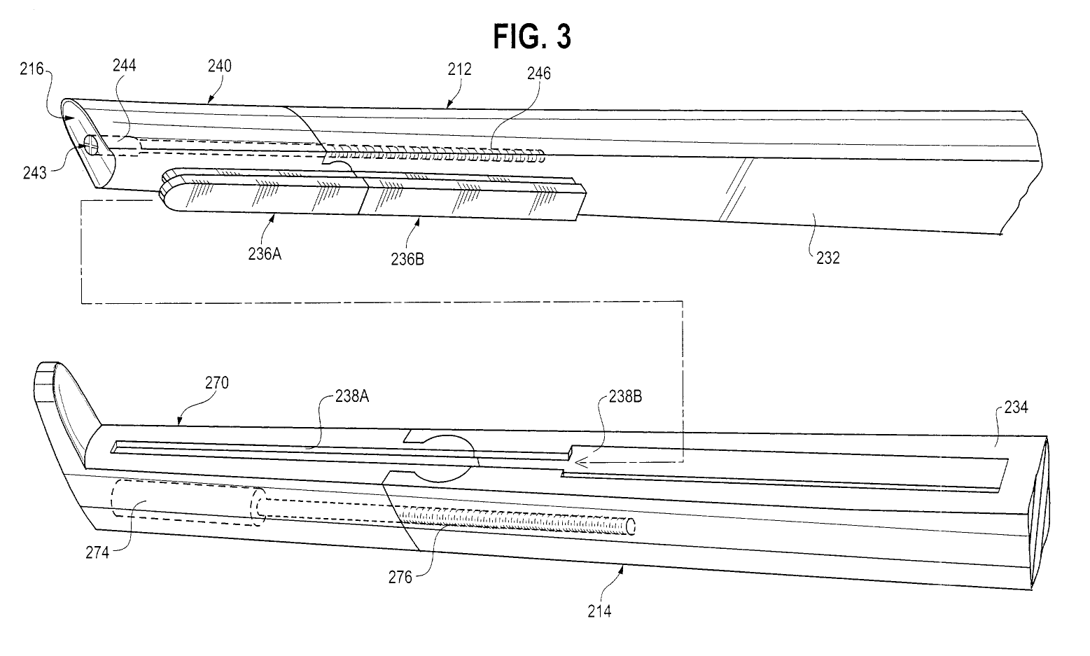

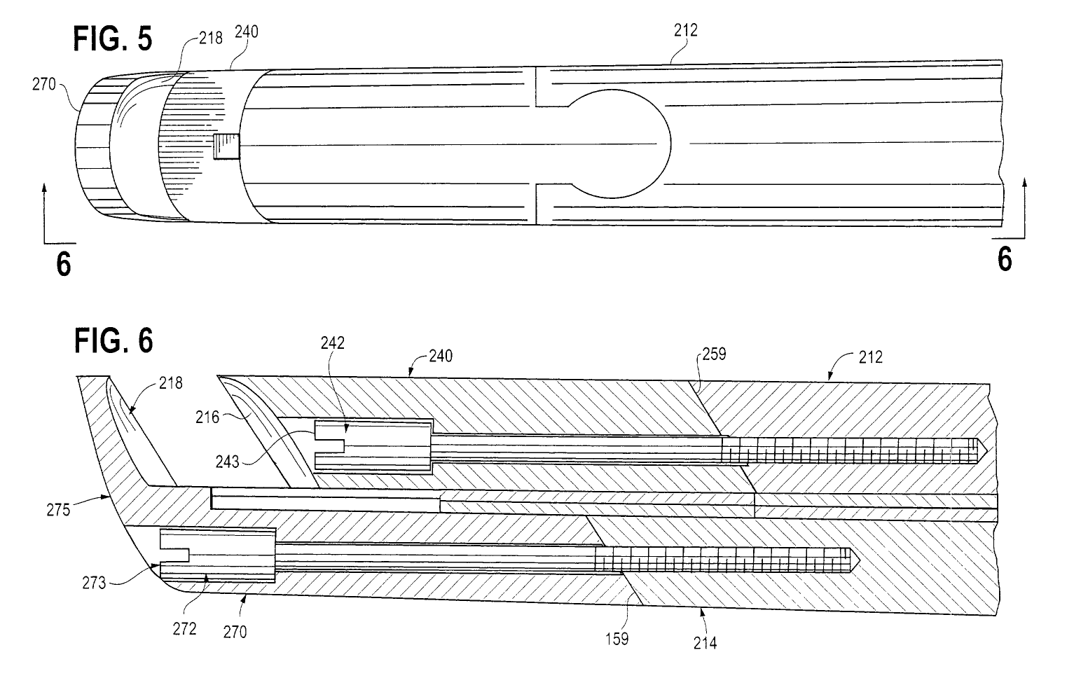

The present invention improves upon this design by providing replaceable tips 240 and 270 as shown in FIGS. 3-6. Tips 240 and 270 each include a cutting section that together effect the tissue cutting function. Arms or base sections 212 and 214 receive and support the replaceable tips. The present invention contemplates that either one of the arms or bases may be configured with a replaceable tip or that both may be so configured. In addition, one or more of the tips may be disposable and removable as well.

As shown in FIGS. 3-6, cutting tips or sections 240 and 270 are attached to arms 212 and 214 through the use of threaded fasteners 242 and 272 which seat within bores 244 and 274 and coact with internal threads 246 and 276. The length of the fasteners and bores may be configured to result in fastener head 243 seating within the bore at a location which allows head 243 to be recessed from the cutting surface 216. Alternately, as shown in FIG. 8, head 243 may also be further configured to conform to the concavity of section 216. This preserves cutting efficiency and prevents tissue and other matter from collecting within the bore. Fastener head 273 may be similarly configured to mimic surface 275 so as to avoid creating a sharp edge on the device and to prevent tissue and other matter from collecting within the bore.

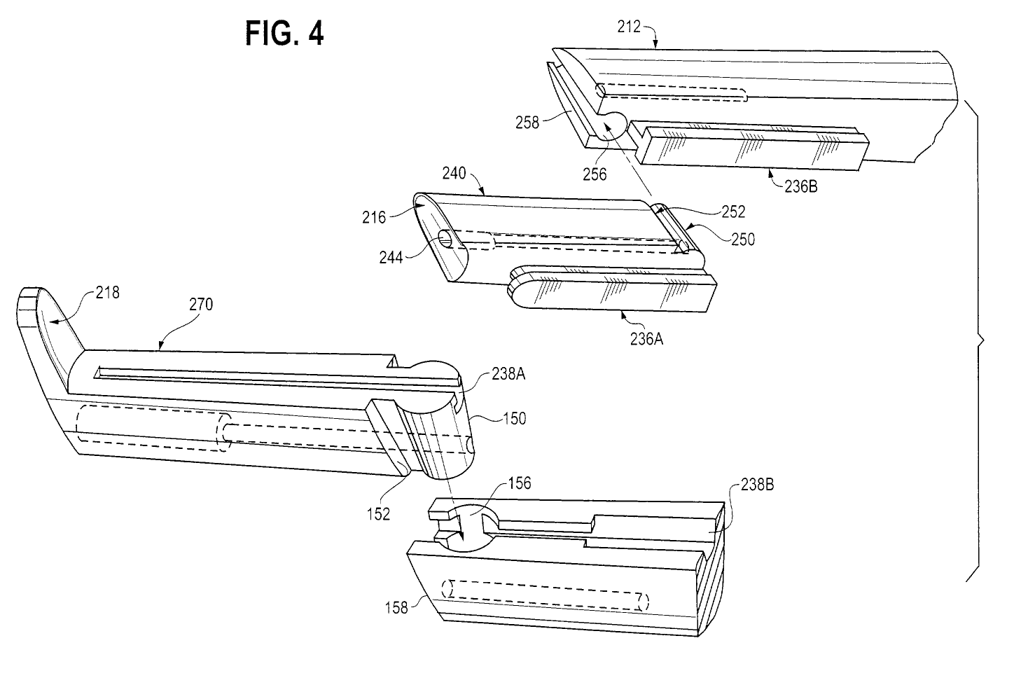

FIGS. 3 and 4 show that cutting sections or tips 240 and 270 may also be configured to incorporate sections of guide 236 and passageway 238. More specifically, tip 240 uses guide portion 236A which aligns with guide portion 236B to form a complete guide. Similarly, tip 270 includes passageway portion 238A which aligns with passageway portion 238B to form a complete passageway.

As illustrated in FIG. 4, section or tip 240 may also include an attachment member 250 that is configured to seat within receiving section 256 on arm 212. Member 250 may be frusto-conical in shape so as to promote a solid attachment but may also be a wide variety of shapes known to those of skill in the art including, but not limited to, cylindrical, circular, and triangular. Likewise, receiving section 256 may be an opening or aperture that is shaped to mate with the attachment member or of such a shape to form a solid and stable fit. With this embodiment, fasteners may be used but are not required since the member and receiving section are interlocking in design.

Tip or section 240 also includes a shoulder 252 that abuts receiving shoulder 258 on arm 212. Shoulders 252 and 258 further assist in the secure and stable attachment of the tip. The shoulders may also be angled relative to the longitudinal axis of arm 212, as illustrated by line 259 in FIG. 6, so as to resist upward forces created when cutting is performed. FIG. 4 also illustrates that section or tip 270 may be attached in a similar fashion. As shown, tip 270 includes an attachment member 150 that is configured to seat within receiving section 156 on arm 214. Member 150 may be frusto-conical in shape so as to promote a solid attachment but may be a wide variety of shapes known to those of skill in the art including, but not limited to, cylindrical, circular, and triangular. Likewise, receiving section 156 may be an opening or aperture that is shaped to mate with the attachment member or to at least form a solid and stable fit. With this embodiment, fasteners may be used but are not required since the member and receiving section are interlocking in design.

Tip 270 also includes an attachment surface or shoulder 152 that abuts or mates with receiving shoulder 158 on arm 214. Shoulders 152 and 158 further assist in the stable attachment of the tip to arm 214. The edges or surfaces may also be angled relative to the longitudinal axis of arm 214, as illustrated by line 159 in FIG. 6, so as to resist downward forces created when cutting is performed. In addition, while cutting sections 216 and 218 are shown in a standard concave, cupped configuration, other shapes may be used as well. Providing a rongeur with the ability to change its cutting characteristics greatly increases the flexibility of the tool over current designs. As mentioned above, the cutting sections may also be disposable.

Alternately, the attachment member and receiving sections may be eliminated or used on just one of the tips. In addition, the edges or shoulders may or may not be angled.

In addition, the present invention contemplates that the stationary arm may include the guides and that the movable arm may include the passageways so as to link the arms in a sliding relationship. Likewise, the cutting tips may include the receiving sections while the arms may include the attachment members.

In use, when the cutting edges wear down, or when a different cutter is desired, the desired tip is replaced by simply unthreading the fastener securing the tip to the base or by disengaging the interlocking member and receiving section. The attachment member is slid out of the receiving section and the procedure is reversed for the installation of a new, sharpened or different tip. Moreover, guide portion 236A will need to be positioned in passageway section 238B prior to removal of the tip.

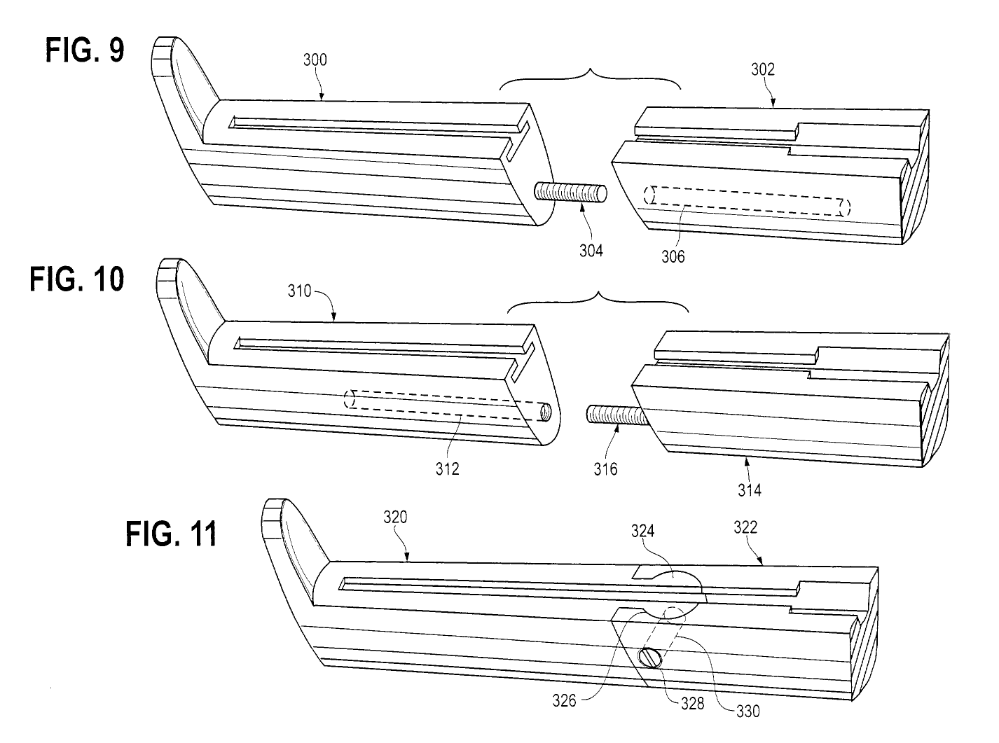

FIGS. 9 and 10 show two alternate embodiments of the present invention. As shown in FIG. 9, the cutting tip or section 300 is secured to base or arm 302 by a threaded rod 304 which extends from tip 300. Threaded rod 304 coacts with internally threaded bore 306 in arm 302 to secure the two sections together.

The embodiment shown in FIG. 10 is of a similar construction with the locations of the threaded rod and threaded bore reversed. Tip 310 contains the internally threaded bore 312 and arm 314 has threaded rod 316. Again the coaction between the threaded elements secures the two components together.

FIG. 11 shows yet another embodiment of the present invention which is similar in design to the embodiments shown in FIGS. 3-5. Tip 320 includes an attachment member 324 that seats within a receiving section 326 in a section of arm 322. A threaded fastener in inserted into a thread bore and transects the receiving section and attachment member to lock the components together. Alternately, a pin may be used instead of fastener 328. Of course, as with the earlier described embodiments, the attachment member may be located on the arm and the receiving section may be located on the tip.

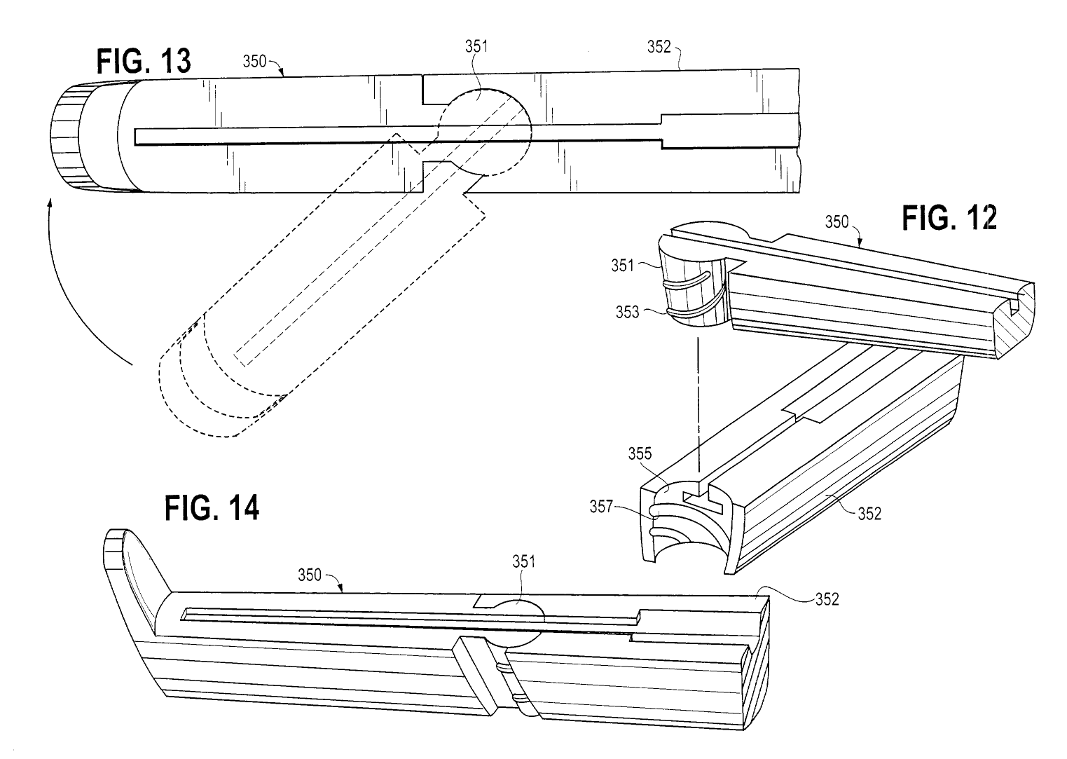

FIGS. 12-14 illustrate an alternate embodiment of the present invention. Tip 350 includes an attachment member 351 having threads 353. As with the earlier described embodiments, arm 352 includes a receiving section 355 that receives the attachment member 351. As illustrated, the receiving section includes internal threads 357. The components are joined together by inserting member 351 into receiving section 355 and rotating the tip so that threads 353 and 357 coact. This coaction joins the sections together. Of course, as with the earlier described embodiments, the attachment member may be located on the arm and the receiving section may be located on the tip.

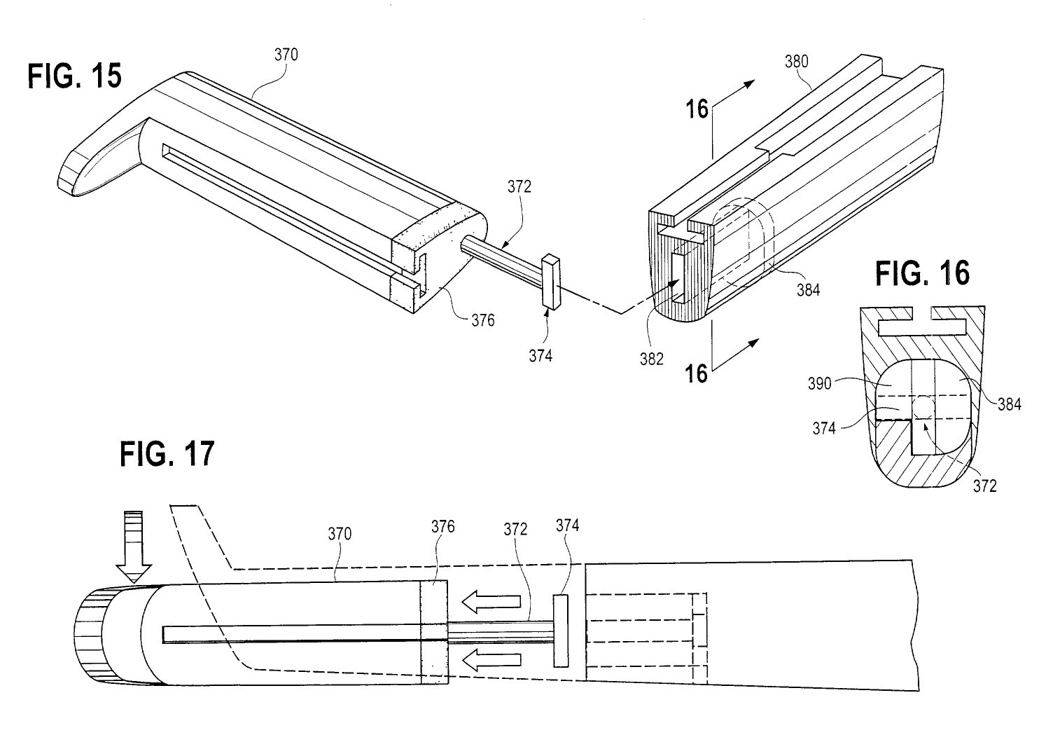

FIGS. 15-17 illustrate yet another embodiment of the present invention. Tip 370 includes an extended section 372 having an enlarged portion 374, which may take the form of a rod and crossbar to form a T-shaped section. A deformable pad 376 is also provided. Arm 380 includes internal bore 382 with enlarged section 384.

The arm and tip are joined together by inserting the T-shaped section into bore 382 and then rotating the tip 90 degrees. This permits cross-bar 374 to engage inner wall 390 and prevents the disengagement of the components. Pad 376 is configured to be slightly compressed when the arm and tip are joined together. This results in a force which biases the cross-bar against the inner wall.

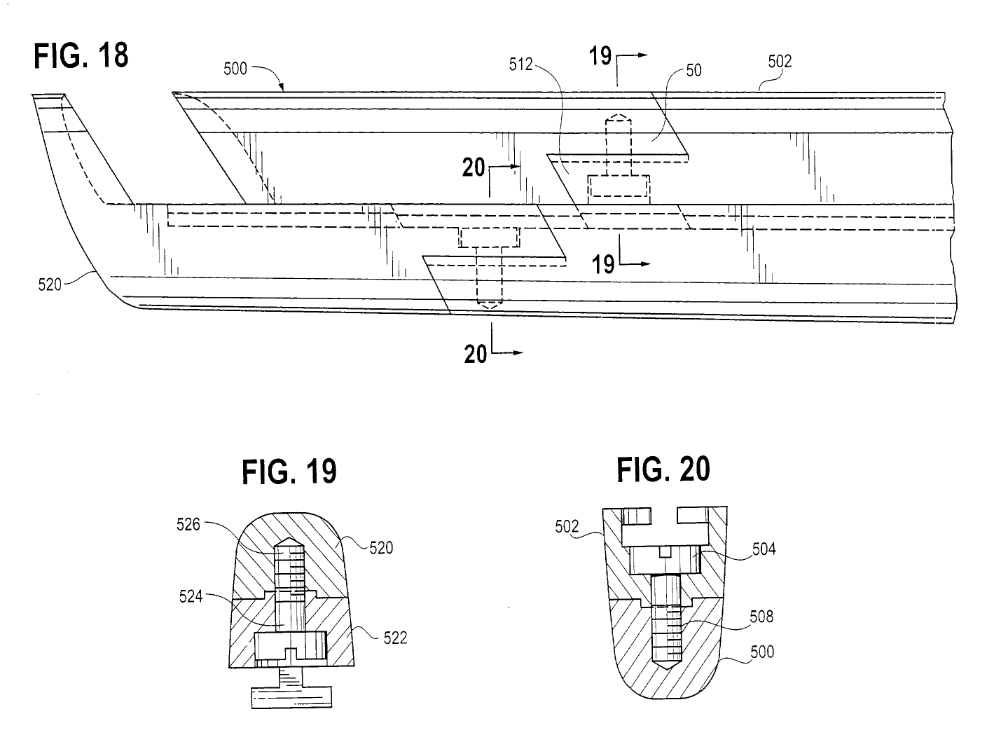

FIGS. 18-20 illustrate an alternate embodiment of the present invention. In this design, tip 500 is secured to base 502 by fastener 504. To accomplish this, tip 500 is configured so that section 510 overlaps section 512 of base 502.

Tip 500 also has internal threads 508, which coact with fastener 504. Of course, the internal threads may be located in arm 502 as well.

Tip 520 is attached to arm or base 522 in a similar fashion through the use of fastener 524 and internal threads 526. Again, the internal threads may also be located in base 522.

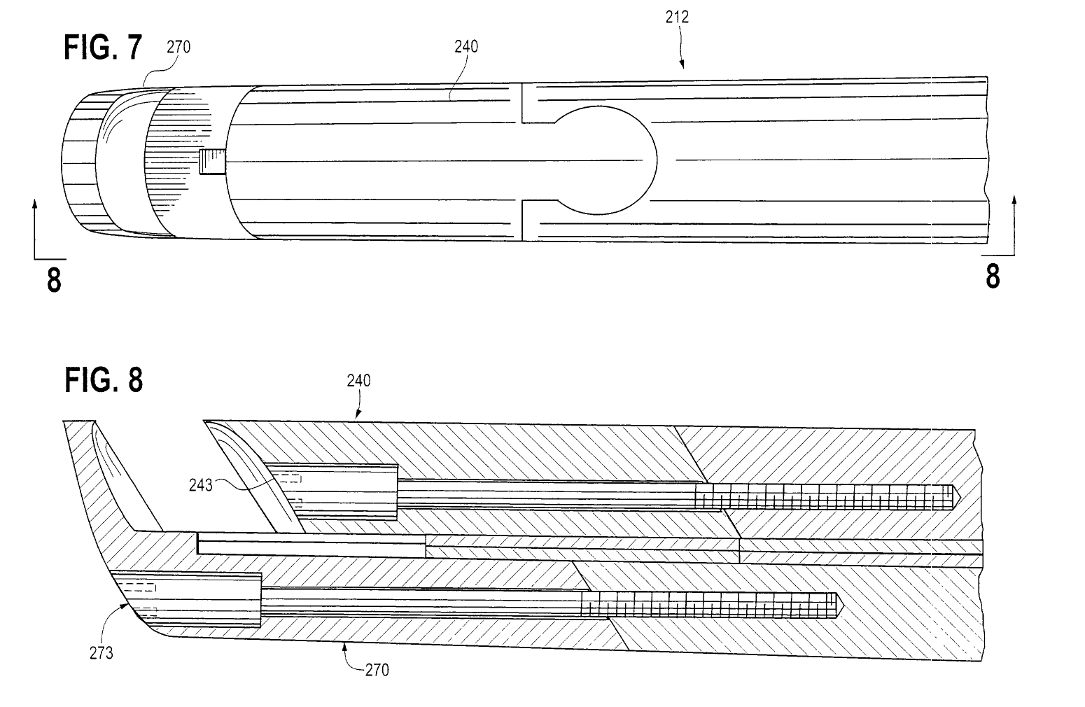

While the preferred embodiments of the present invention have been illustrated and described, it will be understood by those of ordinary skill in the art that changes and other modifications can be made without departing from the invention in its broader aspects. Various features of the present invention are set forth in the following claims. For example, although only the stationary arm is illustrated, the moveable arm may also be configured in the same manner for the embodiments shown in FIGS. 7-20.

What is claimed is:

1. A rongeur comprising:
a moveable arm slidingly connected to a stationary arm;
a stationary handle connected to said stationary arm;
a moveable handle coupled to said moveable arm to permit said moveable arm to operate between open and closed positions;
at least one of said arms adapted to receive a removable cutting tip.

2. The rongeur of claim 1 wherein both of said arms are adapted to receive removable cutting tips, said tips are attached by threaded fasteners which coact with internal threads located in each of said arms.

3. The rongeur of claim 1 wherein one of said arms includes a receiving section configured to receive an attachment member located on said removable cutting tip.

4. The rongeur of claim 1 wherein one of said arms includes a receiving surface configured to abut an attachment surface located on said removable cutting tip.

5. The rongeur of claim 4 wherein said attachment and receiving surfaces are angled.

6. A rongeur comprising:
a moveable arm and a stationary arm linked in a sliding relationship by a pair of spaced apart guides located on one of said arms which seat within a pair of spaced apart passageways located on the other of said arms;
a stationary handle connected to said stationary arm;
internal threads located in said arms;
a moveable handle coupled to said moveable arm to permit said moveable arm to operate between open and closed positions;
at least two removable cutting tips;
a bore located in each of said tips sized to receive a threaded fastener which coacts with said internal threads to secure said tips to said arms.

7. The rongeur of claim 6 wherein each of said arms include a receiving section configured to receive an attachment member located on each of said removable cutting tips.

8. The rongeur of claim 7 wherein said attachment member is frusto-conical in shape and said receiving section is an aperture shaped to mate with said attachment member.

9. The rongeur of claim 6 each of said tips include a receiving section configured to receive an attachment member located on each of said arms.

10. The rongeur of claim 6 wherein each of said arms include a receiving surface configured to abut an attachment surface located on each of said removable cutting tips.

11. The rongeur of claim 10 wherein said surfaces are angled.

12. The rongeur of claim 6 wherein one of said tips includes a guide which seats within a passageway located on the other of said tips.

13. A rongeur comprising:
a moveable arm and a stationary arm linked in a sliding relationship by a pair of spaced apart guides located on one of said arms and a pair of spaced apart passageways located in the other of said arms;
a stationary handle connected to said stationary arm;
a moveable handle coupled to said moveable arm to permit said moveable arm to operate between open and closed positions;
a removable cutting tip having an attachment means to releasably secure said tip to one of said arms.

14. The rongeur of claim 13 wherein said attachment means is comprised of coacting fasteners.

15. The rongeur of claim 14 wherein said attachment means is comprised of external threads located on said tip and internal threads located in said arm.

16. The rongeur of claim 13 wherein said attachment means is comprised of a locking pin which seats within said arm.

17. The rongeur of claim 13 wherein said attachment means is comprised of a pin which transects said arm.

18. The rongeur of claim 13 wherein said attachment means is comprised of a fastener which transects said arm.

19. The rongeur of claim 13 wherein said attachment means is comprised of an attachment member located on said tip and a receiving section located on said arm.

20. The rongeur of claim 13 wherein said attachment means is comprised of an attachment member located on said arm and a receiving section located on said tip

21. The rongeur of claim 19 wherein said attachment member is frusto-conical in shape and said receiving section is an aperture shaped to mate with said attachment member.

22. The rongeur of claim 19 wherein said attachment member is frusto-conical in shape and said receiving section is an aperture shaped to mate with said attachment member.

23. The rongeur of claims 19 and 20 wherein said attachment member and receiving section are threaded.
